SEQUENCE LISTING (1 sequence)

SEQ ID NO: 1 - DNA, 25 bp, Artificial Sequence
  misc_feature: this sequence has an aminated  3′-terminus
gggagagccatagtggtctgcggaa

(19) United States
(12) Patent Application Publication
(10) Pub. No.: US 2004/0115685 A1
(43) Pub. Date: Jun. 17, 2004

(54) Immobilisation of ligands

(76) Inventors: Juan Carlos Tercero, Madrid (ES); Lucia Garcia, Madrid (ES); Jose Manuel Guisan, Madrid (ES); Roberto Fernandez, Leganes (ES); Manuel Fuentes, Salamanca (ES)

Correspondence Address: FISH & RICHARDSON PC, 225 FRANKLIN ST, BOSTON, MA, 02110, US

(21) Appl. No.: 10/471,351

(22) PCT Filed: Mar. 8, 2002
(86) PCT No.: PCT/GB02/01059

(30) Foreign Application Priority Data
Mar. 8, 2001 (GB) 0105745.4

(51) Int. Cl. C12Q 1/68; C07H 21/04

(52) U.S. Cl. 435/6; 536/23.1

(57) ABSTRACT

Oligonucleotide is immobilised to a surface using a saccharide or related polymer with at least two different functionalities.

The present invention relates to the immobilisation of ligands, and more particularly the immobilisation of DNA.

### BACKGROUND OF INVENTION

The most widespread DNA hybridisation technique in liquid media with microtitre plates uses streptavidin coated plates. The biotinylated DNA probe is immobilised at the surface by a biotin-streptavidin bond. This bond is very strong and quick to form, but is unable to resist extreme hybridisation conditions.

The detection by hybridisation of DNA molecules with specific mutations in their sequence requires extreme conditions during hybridisation. These extreme conditions include temperature, salts and solvents. In such conditions, the streptavidin-biotin DNA probe complex breaks or is desorbed from the plate. There is a need for the development of a new system for immobilising DNA through covalent bonding of DNA to active groups on the surface of the plastic of the microtitre plates able to withstand these conditions necessary for highly astringent hybridisation.

Commercially available plates such as DIAPOPS, NUCLEOLINK or COVALINK (NUNC) use secondary amino groups for immobilisation of phosphorylated DNA probes at the 5′ terminus. However, despite the fact that the manufacturers give their assurance that immobilisation occurs in this fashion, the DNA probe is not recovered after treatment with acid (which hydrolyses the immobilisation bond), the immobilisation yield is very low, and the resulting immobilised DNA does not permit highly sensitive and specific experiments of hybridisation.

The simplest protocol for activation of the amino groups is their modification with dialdehydes (for example, glutaraldehyde). This allows subsequent reaction of the activated plates with aminated DNA probes. Bifunctional reagents such as NHS or EDC that contain secondary amino groups (commercial NUNC plates) have also been used for immobilisation of DNA at the surface,

However, these conventional protocols have many important limitations for immobilisation of any type of macromolecule, and in particular, for immobilisation of DNA probes, namely:

(a) the chemical reactivity between the aminated ligands and the activated plates is usually relatively low and so the efficiency and sensitivity of the resulting plates is low;

(b) the DNA probes are immobilised near to the surface of the plate, which could lead to steric hindrance of the hybridisation between the immobilised probe and the free DNA product of the PCR amplification;

(c) many amino groups in the support may not react, also the amino-aldehyde bonds generate ionisable secondary amino groups. Both groups can promote undesired non-specific absorptions of DNA to the plates.

Thus, while in principle, the commercially available plates constitute the best starting point for the covalent immobilisation of biomacromolecules, there are problems. The commercial plates usually have amino groups with a very high pK. As a result, these amino groups are in principle not very reactive for their activation (for example, with poly-aldehydes) and, moreover, show extraordinary properties (undesirable in this case) as anion exchangers. It is thus necessary to greatly increase their reactivity and at the same time reduce as far as possible their capacity for anion exchange (non-specific absorption of oligonucleotides).

Commercial DNA probes can be obtained with a primary amine residue added to one of the 3′ or 5′ termini of the oligonucleotide. To optimise the hybridisation process, probes are manufactured in which the primary amine group is separated from the oligonucleotide by relatively large spacer arms (C5-C7) consisting of linear aliphatic chains. However, the amine groups bound to these long aliphatic chains have rather high pKs and are not very reactive with the aldehyde groups of the probe. One way of optimising the reactivity of these rated probes would be by performing the immobilisation at a very alkali pH. The problem is that this would cause damage because of the low stability of the aldehyde groups of the plates and because of possible undesired participation by the amino groups of the bases of the oligonucleotide in the immobilisation process.

The alternative is to use commercial probes with shorter spacer arms (C2-C3), in which case the reactivity of these amines increases considerably. However, use of very short spacer arms leads to a considerable increase in the steric hindrance of the hybridisation between immobilised DNA and free DNA.

When the surface to be used for the immobilisation of DNA probes or other ligands is very inert and does not possess any ionised groups, the simple use of homofunctional-dextrans (J. Biochem. Biophys. Meth. (1985) 10, 321-328; Nuc. Acid. Res (1987) 15, 13, 5373-5390; WO 98/22620; WO 94/03530) may be enough to attain an adequate immobilisation of the biomolecules. The use of these polymers makes possible the introduction of large numbers of aldehyde groups for every group that can be activated on the plate. In addition, these polymers provide spacer arms necessary to facilitate the interaction between the biomacromolecules and the immobilised ligands. However the major drawback of these methods is a high non-specific adsorption of the DNA to be hybridised, due to the positive charges remaining in the surface. This disadvantage is reduced by using prehybridisation steps or large quantities of exogenous DNA to block the non-specifc adsorpion of the DNA complementary to the probe. The present Example 1 and FIG. 1 show the drawbacks of these methodologies.

### SUMMARY OF THE INVENTION

According to the present invention, we provide an oligonucleotide such as DNA immobilised to a surface by a hetero-poly-functional saccharide or related polymer.

The invention also provides a procedure for immobilisation of DNA or other oligonucleotide probes using modified hetero-poly-functional saccharides.

In the invention, the polysaccharide or other polymers act as long, inert and hydrophilic spacer arms in which non-specific binding of ilogonucleotide is avoided.

The invention further provides method of detection using the immobilised oligonucleotide, typically by contacting with a sample and assessing hybridisation.

### DETAILS OP THE INVENTION

For convenience, the details will be described in relation to DNA, though the invention can be used with other oligonucleotides including RNA or PNA.

The solution that we propose is to use hetero-funcional dextrans or other polymers, preferably hydrophilic polymers, as spacer arms to avoid steric hindrance, having hetero functional groups on the one hand for immobilisation of DNA, such as aldehyde groups, and on the other hand to avoid the non-specific adsortion of the DNA of the sample, such as negative charged groups. After the immobilization the DNA probe has a freedom comparable to the soluble DNA. This solution may be reinforced by a final blocking of primary amino groups via amide bonds with aliphatic groups.

The polysaccharide of this invention is thus hetero-poly-functional, and is typically based on a natural or synthetic polysaccharide, including derivatives thereof. Suitable saccharides include dextrans, carboxymethyl dextrans, hydroxyethyl- and hydroxypropyl-starches, glycogen, agarose derivatives, hydroxyethyl- and hydroxypropyl-celluloses, carragenates, amylose, pectin and natural gums and derivatives and synthetic polymers having nucleophilic functional groups, as polyvinyl alcohols, polyallyl alcohol, polyethylene glycols and substituted acrylates.

The preferred saccharide is a dextran.

In a preferred aspect, the invention provides a procedure for immobilisation of DNA probes based on the use of hetero-poly-functional polymers that contain aldehyde or other reactive groups for immobilisation of DNA molecules and negatively charged groups (for example carboxy groups, sulphate groups or phosphate groups) to improve the immobilization of the polymer and for blocking the positive charges on the support responsible for non-specific adsorption of DNA.

The procedure for immobilisation of DNA probes can be based on the use of several steps where the aminated plates initially are modified with hetero-poly-functional polymers of a high molecular weight (such as dextran between 20,000 and 40,000,000 Daltons) containing a low concentration of groups with negative charge and subsequently performing a second modification with polymers of lower molecular weight (such as dextran between 6,000 and 340,000 Daltons) but containing a high concentration of negative groups to optimise the block of the positive charges at the surface of the aminated plates.

The use of acetic anhydride or other similar reagent might substitute the use of the second smaller dextran, reducing the non-specific adsorption. In this way, the anhydride blocks the primary amino groups remaining in the surface and the aspartic dextran blocks the secondary amino groups, eliminating the non-specific adsorption of the DNA form the sample.

In one aspect, the spacer arm is a biopolymer that can be activated sequentially such that its modification for example with aspartic acid, its immobilisation on the microtitre plates or other surface and its subsequent reaction with the aminated DNA probes can be designed separately.

In a related aspect, the invention is based on the use of DNA probes with amino groups at their 3′ or 5′ termini connected to the DNA sequence by a short linker that increases the yield of immobilisation.

The invention enables performing experiments of hybridisation of DNA immobilised on mictrotitre plates or other surfaces, with free DNA in the presence of different concentrations of formamide (from 5 to 50%) to increase the specificity of the hybridisation.

The immobilisation of DNA probes and of any other biopolymer or aminated ligand can be carried out using poly-functional aldehyde-dextrans or other hetero-poly-functional polymers to activate different surfaces of interest in diagnostics, such as microtitre plates, magnetic particles, agarose supports, epoxy-acrylic resins, nylon, glass surfaces, gold surfaces, etc.

A general method for activating the surfaces is to aminate them (by introducing a small concentration of amino groups with low pK) and subsequently perform a modification with hetero-poly-functional polymer of this invention, such as an aldehyde-dextran.

In all cases, the polymers such as dextrans can be oxidised to allow reaction with the amino groups of the DNA probes or with amino groups of other ligands of interest in diagnostics, in genomics and in proteomics.

The solution propose in this invention is the use of short (instead of long) aliphatic linkers between the amino group and the 3′ or 5′ end of the oligonucleotide, increasing the reactivity of the amino group and increasing the yield of immobilisation. This is possible because now the dextran or other polymer is going to act as spacer arm.

The use of heterofunctional aspartic-aldehyde dextrans permits an easy covering of the aminate surface via physical adsorption, follow by the covalent amino-aldehyde reaction. Aldehyde-dextran features make difficult the direct amine-aldehyde reaction when the pK of the amine in the support is high.

The dextrans-aldehydes can be used for activating surfaces containing carboxyl groups, epoxide groups, hydroxyl groups, etc. In the most complex case (plastic plates not resistant to organic media), all the chemical modifications can be carried out in aqueous medium.

Covalent bonding for the immobilisation of a DNA probe that we present is acceptable because the following conditions are met:

a.—there is a very stable chemical bond resistant to pHs, temperatures and the presence of salts, solvents and nucleophiles in the hybridisation mixtures,

b.—spacer arms to facilitate the hybridisation and allow the immobilisation of a larger amount of probe

c.—very inert supports, especially regarding electrostatic absorptions of DNA (a poly-anion with a high capacity for absorption on cationic surfaces)

d.—immobilisation of the probe by means of a single bond via the termini of the DNA probe, which allows hybridisation with the entire sequence of bases of the probe.

The present method can be used for immobilisation of DNA and other macromolecules at any surface (microtitre plates, magnetic particles, etc), which contains, for example, amino; hydroxyl, carboxyl or epoxide groups that can be activated. By way of example, the most complex and frequent problem is described in detail, namely the use of commercial aminated microtitre plates for binding DNA probes or other ligands containing amino groups.

In the preferred embodiments of this invention, the use of hetero-poly-functional polymers (with anionic and aldehyde groups) of very high molecular weight is proposed for the modification of aminated microtitre plates. These polymers contain carboxyl groups and aldehyde groups, which gives them excellent properties for the activation of aminated plates. These beneficial properties include:

a.—These hetero-poly-functional polymers react very easily with the aminated plates, even though they possess amino groups with a high pK. Initially, the polymers are absorbed ionically to the plate and then their covalent attachment takes place by amino-aldehyde reaction between neighbouring groups (absorbed polymer and the plate)

b.—These large polymers, absorbed ionically and covalently, to the plates have very positive effects reducing non-specific ionic interactions between negative charges of the DNA and the positive charges of the Go groups of the aminated commercial plates.

c.—These poly-aldehyde polymers improve the immobilisation of the oligonucleotide probes as they allow the generation of a large number of aldehyde groups for each amino group initially present on the aminated commercial plate.

d.—These hydrophilic polymers are excellent spacer arms to facilitate the hybridisation between the immobilised DNA arms and the free DNA, as they convert a surface into an accessible volume for the hybridisation. As we mentioned earlier, the synthetic probes with amino groups bound to the 3′ or 5′ termini of the oligonucleotide by small spacer arms have a good reactivity (low pK of the amino group) but generate steric hindrances for the hybridisation experiments. The use of dextrans as spacer arms completely eliminates these hindrances and allows the use this type of probe.

In many cases, it is possible to use polysaccharides with certain groups (for example, of ionic nature) already incorporated by nature (carragenates, etc). Most sugars can be activated by one of the methods described. In other cases, we can use mono-functional polymers, such as the aldehyde dextrans for example, and introduce the desired groups in a controlled fashion, where such groups include negatively charged groups such as the carboxylic acids, phosphates, sulphates, amino acids, amines, etc). The polymer molecules are very large and have many groups that can be activated by many different methods described in the scientific literature. If these modifications were used partially, for example, sequentially affecting small percentages of the modifiable groups, we can obtain poly-functional polymers made to measure.

By way of an example, which is in no way limiting, of the strategies that can be used within the framework of this patent, we will describe the preparation of poly-aspartic/poly-aldehyde dextran and its use for activating supports with aminated surfaces.

a.—The oxidation of dextran molecules is a reaction with a well defined stoichiometry: each glucose molecule consumes two molecules of periodate and produces two aldehyde groups. If less periodate is added than is necessary to completely oxidise the glucose of the polymer, partial and completely controlled modifications can be attained.

b.—Our research group has designed different methods that allow the transformation of these aldehyde groups with aminated compounds, such as ethylenediamine or amino acids, thus managing to introduce a molecule of these compounds for every glucose molecule of the dextran. For example, the incubation at pH 8 and in the presence of trimethyl-aminoborane of dextran-aldehyde in sodium aspartate 1.5 M allows quantitative modification of the aldehyde groups of the dextran molecule. After reducing with borohydride, the aldehyde-amino links will be converted into very stable bonds, and any unreacted aldehyde will be converted into inert alcohols. If this dextran molecule had just 20% of its glucose molecules oxidised, we could obtain a polymer with 80% of intact glucose and 20% of glucose modified with aspartic groups.

c.—The previous polymer can be submitted to another partial oxidation of the glucose molecules, obtaining a poly-aldehyde/poly-aspartate.

d.—If this is incubated with a solid that possesses amino groups (for example, plates or magnetic particles, or any other aminated surface) in conditions in which the support surface has a certain positive charge, the polymer will absorb to the surface, increasing the concentration of aldehyde and primary amino groups. The reactivity of these groups will also be increased, accelerating the covalent immobilisation of the polymer at the surface. After reducing with borohydride, the amino-aldehyde links will be transformed into highly stable bonds, and the aldehydes into inert alcohols.

e.—The support with the immobilised polymer is submitted to oxidation with periodate 50 mM, generating aldehyde groups. Thus, we have an activated plate ready for use.

In principle, most of the chemical systems described in the scientific literature for the immobilisation of proteins are valid for immobilising poly-functional polymers on the different supports: microtitre plates, magnetic particles, epoxy-acrylic resins, agarose gels, silica, glass, nylon, etc. As indicative, but not limiting, examples, of chemical systems that could be used for the immobilisation of polymers on the different supports, we now mention some of the possibilities for activating the support:

a.—amine groups. Aminated supports can be made to react directly with activated polymers with any of the groups described in the literature that can react with amino groups (for example, poly-aldehydes, poly-epoxides).

b.—carboxyl groups. This type of group can be totally or partially modified (allowing surfaces to be obtained with net negative charge or even net positive charge) with ethylenediamine and carbodiimide to obtain an aminated support, which can react as in the previous case. The carboxyl groups will also be able to react with aminated polymers directly if the pK of the amino group is sufficiently low (for example, dextran-ethylenediamine)

c.—epoxide group. The epoxide supports can be used to directly immobilise polyols (for example, by incubation at pH 12) or thiolated or aminated polyols. They can also be modified with diamines to obtain an aminated support (for example, by incubation with ethylenediamine 1 M at pH 11) and used as in case (a). After acidic or alkali hydrolysis, they could be oxidised with periodate to obtain aldehydes, which, in turn, can be modified with diamines or immobilised aminated polymers.

d.—hydroxyl groups. This type of group can be made to react with epichlorohydrin at alkali pH to transform the hydroxyl groups into epoxide groups, giving case c once again.

e.—silicates, aluminates, glass, etc. These solids present silanols that can be activated with tri-methoxysilanes derivatised with amino or epoxide groups for general mated supports or epoxides that are used according to the previously described protocols.

The possibilities of activating polysaccharides for immobilisation on solids are wide-ranging. As indicative, but not limiting, examples, of the chemical systems that could be used for the activation of polymers for immobilisation on the different supports, we mention the following as some of the simplest examples:

a.—Preparation of poly-aldehydes. Most polysaccharides can be oxidised with periodate to give aldehyde groups. For example, in the case of dextran, two molecules of periodate are consumed for every glucose molecule to yield two aldehyde groups. Using dextrins (of low molecular weight so that they are soluble in water) a molecule of periodate directly produces two aldehyde groups. These polyaldehydes can react with aminated supports, and after reducing with borohydride, remain bound to the solid by secondary amino bonds (very stable), thus generating a completely inert polyol.

b.—Preparation of poly-epoxides. The polysaccharides can be partially modified with epichlorhydrin at alkali pH to introduce epoxide groups from the alcohols. These epoxide groups could be made to react with aminated supports, generating an activated support with epoxide groups.

c.—Preparation of poly-amines. The two previous polymers can be modified with ethylenediamine, thus generating an aminated polymer with primary amino groups of low pK, ideal for reacting with epoxide or aldehyde supports.

Immobilisation of DNA probes on supports activated with hetero-poly-functional dextran can be tailored according to the DNA.

a.—Immobilisation of small oligonucleotides (say 12-20 bases)

Commercial DNA probes are used modified with primary amino groups. Two different types of probe are typically used:

i.—Probes with primary amino groups separated from the oligonucleotide by a long spacer, arm (say C7).

This probe appears to be appropriate for directly immobilising on activated surfaces because its relatively long spacer arm favours the process of hybridisation between the immobilised probe and the soluble PCR products. However, the amino group has a very high pK (approximately 10.5-11) and so it is very difficult to immobilise.

ii.—Probes with primary amino groups separated from the oligonucleotide by a very short spacer arm (say C3). For example:

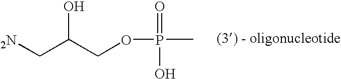

The amino group of this probe has a much smaller pK (8.0-9.0) due to the proximity of the phosphodiester as well as its beta hydroxyl group. Therefore, it is much more reactive than the amino group mentioned in the previous case and is immobilised more easily on plates activated with aldehyde groups. The possible problems of steric hindrance of hybridisation should not be important as the probe binds to the dextran molecules, which act as spacer arms.

b.—Immobilisation of long DNA molecules (say larger than 40 base pairs) generally obtained by PCR

The molecules of DNA that are to be immobilised on the plate will be aminated beforehand with different commercial reagents ot the type

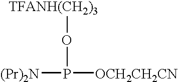

3-(trifluoroacetylamino)propyl-(2-cyanoethyl)-(N,N-diisopropyl)-phosphoramidite

Thus, the probes are modified with amino groups of low pK and they will be made to react with the aldehyde groups of the dextrans bound to the probe.

However, the most useful method for obtaining long probes, mainly of cDNA will be synthesis by reverse transcription from the cellular mRNA or clones that contain it, with subsequent amplification by PCR using one of the primers with an NH₂ group at the 5′ terminus.

The DNA molecules labelled at their 5′ terminus by any of the previous methods can be immobilised on surfaces using the same methods as for oligonucleotides described in this patent.

c.—Immobilisation of DNA Analogs

Other analogs of DNA such as RNA or PNA are also suited for immobilisation using the methodology of the present invention.

In this invention, a series of original contributions are made to the covalent immobilisation of DNA probes and other ligands on surfaces of interest in diagnostics (for example, microtitre plates, magnetic particles, etc.):

1.—Use of dextrans or other polymers of a very high molecular weight to increase the amount of useful space for immobilisation of the probe (conversion of a two-dimension surface into a volume)

2.—Use of dextrans or other polymers as spacer arms to facilitate the hybridisation between the immobilised probe and the PCR products.

3.—Immobilisation of large hetero-polyfunctional polymers such as dextrans-aldehyde-carboxyl on aminated plates of high pK (quick prior physical absorption followed by a quick covalent immobilisation, leading to an increase in the rate and yield of immobilisation)

4.—A second modification to the plate with smaller highly charged polymers, such as dextrans with high concentration of aspartic residues to neutralise possible electrostatic interactions between the immobilised oligonucleotide or the soluble DNA to be tested and the plate. Acetic anhydride can fulfil a similar function, as noted above.

5.—A second activation of the polymer, for example dextran, bound to the plate for covalent bonding of the probe, but means of a single very stable bond between the probe and the plate and localised at the 5′ or 3′ termini of the oligonucleotide without affecting the amino groups of the oligonucleotide bases.

6.—The use of dextrans or other polymers as spacer arms allows us much greater versatility in the choice of aminated oligonucleotide probes that we can use in the immobilisation. Thus, for example, we do not require spacer arms in the probe, we can use aminated probes at the 3′ or 5′ terminus of the oligonucleotide whose amino group is a short distance from the oligonucleotide. These amino groups have a relatively low pK and so are very reactive towards dextrans-aldehyde of the plate. Thus, the immobilisations of the aminated probe can be carried out in very selective and gentle conditions where the amino groups introduced at the terminus of the probe reacts easily with the aldehyde groups of the dextrans, while the amino groups of the bases of the oligonucleotide sequence remain unaltered without reacting with the aldehyde groups of the immobilised dextrans on the plate.

In the preferred embodiments, we achieve the following advantages, among others:

a.—activation of epoxide groups can be performed by reacting with ethylenediamine at alkali pH.

b.—the carboxyl groups can be activated with ethylenediamine, but in this case, at acidic pH and in the presence of carbodiimide.

c.—the hydroxyl groups can be activated with epichlorohydrin at alkali pH, thus forming epoxide groups on the support which are activated as described in a.—.

The supports containing epoxide groups can also be derivatised with dextrans without the need for prior amination. The epoxide groups react with the hydroxyl groups of the dextrans at very alkali pH, using borohydride as a reducing agent to prevent oxidation of the dextrans at these alkali pHs. Finally, the remaining epoxy groups may be blocked or destroyed.

### FIGURES OF THE INVENTION

FIG. 1 illustrates the prior art

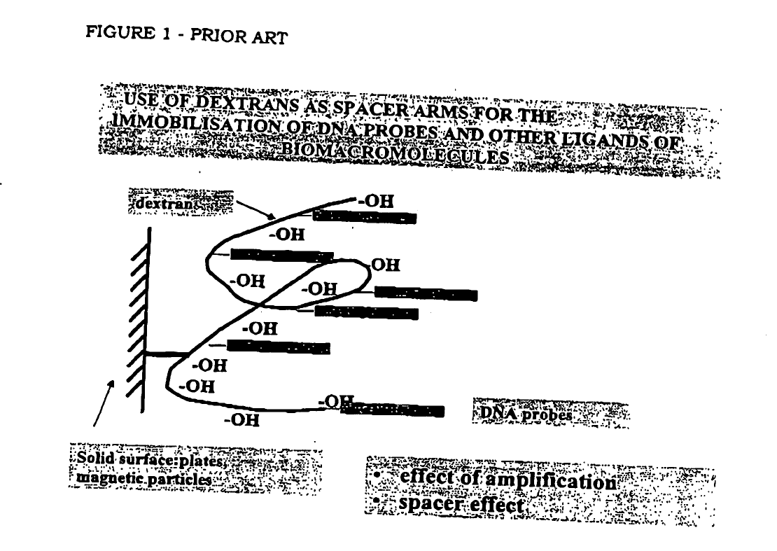

FIG. 2 illustrates the present invention

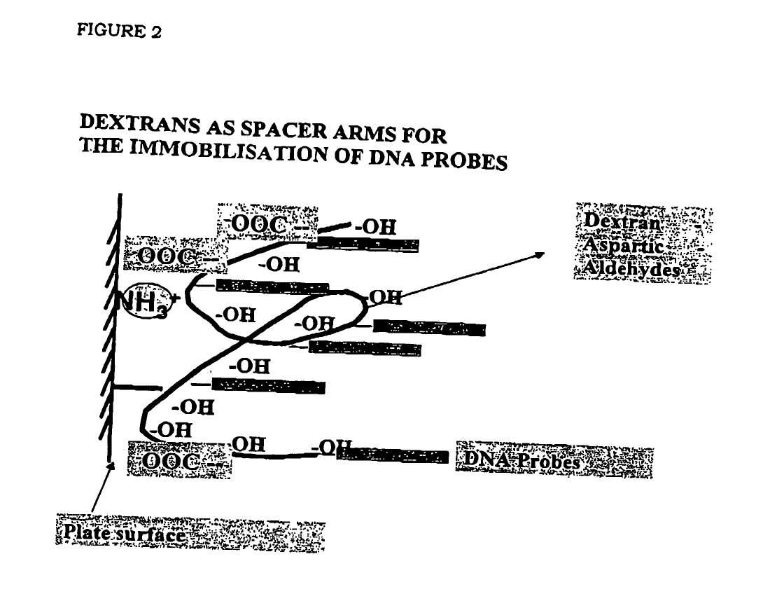

### EXAMPLES OF THE INVENTION

By way of example, we will describe the most complex immobilisation protocols: the immobilisation of aminated DNA probes on microtitre plates aminated with amine groups of high pK (not very reactive and responsible for non-specific interactions with soluble DNA)

### EXAMPLE 1

Immobilisation of Aminated Oligonucleotides on Aminated Plates Activated with Dextran of 2,000,000

Preparation of Aldehyde-Dextran:

Solid sodium periodate (0.872 g) is added to a solution of dextran (1.67 g of dextran in 50 ml of water, Molecular weight 2×10⁶). The mixture is stirred (with a magnetic stirrer) in order to dissolve the periodate. The reaction of oxidation of dextran is then allowed to proceed for 2 hours at room temperature. Once the reaction has finished, the solution is dialysed 7 times against 5 litres of distilled water in order to eliminate the periodate reagent and the side product of the reaction: formaldehyde. We thus obtain a dextran with 20% of the glucose molecules present in the polymer oxidised as di-aldehydes.

Modification of the Aminated Plate with Aldehyde Dextran

To 20 ml of aldehyde-dextran, 1 ml of phosphate buffer 500 mM pH 7 is added followed by trimethylaminoborane in powder form until a concentration of 150 mM is reached. This solution is stirred with a magnetic stirrer for 45 minutes. Then, 350 μl are added to each well of an aminated plate (for example, those supplied by Costar), and they are left to react for 24 hours at room temperature. After this time, the plate is emptied and dried by holding it upside down and tapping it over filter paper. Finally, the bonds formed between the dextran and the plate are reduced (forming very stable secondary amino bonds), along with the remaining aldehyde groups (forming very inert hydroxyl groups) by adding 350 μl of carbonate buffer, 500 mM, pH 10.5, containing 10 mg/ml of sodium borohydride, to each well. After 30 min, the plates are washed with plenty of water, and dried by holding them upside down and tapping them over filter paper.

Immobilisation of the Aminated Oligonucleotide.

The dextran immobilised on the plate is oxidised once more by adding 100 μl of sodium periodate 50 mM to each well. After 1 hour at room temperature, the plates are washed with plenty of distilled water. Next, 15 μl of phosphate buffer 65 mM, pH 7.0, containing 369 ng of probe aminated at the 3′ terminus and NaCl 330 mM, are added to each well. The immobilised dextran and the probe are allowed to react by incubating the plates, sealed with adhesive paper, in an oven at 55° C. for 72 hours with gentle shaking. After this time, thc plates with the immobilised probe are dried by holding them upside down and tapping them over filter paper, and reduced by adding 350 μl of sodium borohydride (100 mg/ml) in carbonate buffer 500 mM, pH 10.5, and letting stand for 1 hour at room temperature. Finally, the plates are washed with distilled water and dried by holding them upside down and tapping them over filter paper.

Results of Hybridisation

Using the protocol described in the previous sections, a specific probe of the Hepatitis C virus was immobilised in aminated microtitre plates. The probe was aminated at its 3′ terminus (5′-GGG AGA GCC ATA GTG GTC TGC GGA A-3′-C3-NH₂). The plates were used for the detection by hybridisation of a fragment of the amplification of the RNA of the Hepatitis C virus that contained the complementary sequence of the probe. 15 μl of the amplified product, labelled with dUTP-digoxigenin, obtained from a clinical sample containing approximately 50 ng/μl of DNA and 1:5 serial dilutions thereof, were assayed. The hybridisation was performed in wells that contained 100 μl of hybridisation buffer (TrisHCl 5 mM, pH 7.5, 1 mM EDTA, 1×SSC and 2× Denhardt's) for 1 hour at 50° C. After this, the hybridised DNA was evaluated by detection of the labelled digoxigenin that had been incorporated using detection reagents and conditions described by the manufacturer (Roche). Finally, the absorption at 405 nm was measured. The results obtained are shown below.

| | Signal | |
|---|---|---|
| Dilution of amplificate | Commercial plate | New plate |
| 1/25 | 3.46 | 1.9 |
| 1/125 | 3.303 | 1 |
| 1/625 | 1.333 | 0.6 |
| 1/3125 | 0.8 | 0.2 |
| Non-complementary DNA (1/25) | 0.18 | 1.353 |

We can therefore conclude that the probe immobilised on the plate is able to hybridise with its complementary DNA, detecting quantities of DNA of 1.2 ng/μl, fivefold lower than the streptavidin-Biotin commercial plate. However, when incubation is performed with amplified labelled DNA that does not contain the complementary sequence, at the high concentration of 30 ng/μl, is almost as big as the signal obtained with the complementary DNA. This indicates that there is a very high non-specific retention of the DNA of the sample due to ionic interactions with the support The use of microtiter plates as described in this example in diagnostic procedures with clinical samples will lead to false-positive results. Example 2 and 3 describe the elimination of these nonspecific interactions.

### EXAMPLE 2

Immobilisation of Aminated Oligonucleotides on Aminated Plates Activated with Aspartic Dextran of 2,000,000

Preparation of Aspartic-Aldehyde-Dextran:

Solid sodium periodate (0.872 g) is added to a solution of dextran (1.67 g of dextran in 50 ml of water, Molecular weight 2×10⁶). The mixture is stirred (with a magnetic stirrer) in order to dissolve the periodate. The reaction of oxidation of dextran is then allowed to proceed for 2 hours at room temperature. Once the reaction has finished, the solution is dialysed 7 times against 5 litres of distilled water in order to eliminate the periodate reagent and the side product of the reaction: formaldehyde. We thus obtain a dextran with 20% of the glucose molecules present in the polymer oxidised as di-aldehydes.

The oxidised dextran is mixed with an equal volume (50 ml) of a solution of sodium aspartate 3 M at pH 7.5 containing 200 mM of trimethylaminoborane. The reaction mixture is kept under vigorous magnetic stirring for 15 hours. After this time, 5 ml of carbonate buffer, 500 mM, pH 10.5, is added, containing 100 mg/ml of sodium borohydride. This mixture is incubated for 30 minutes with magnetic stirring at 20-25° C. The excess sodium borohydride is then destroyed by lowering the pH of the reaction mixture to 6.00 using concentrated hydrochloric acid. The dextran, modified with aspartic acid and reduced, is dialysed 10 times against distilled water at a ratio of approximately 1/50.

The dextran is oxidised once again at 20% with 0.872 mg of periodate, stirring the ire for 2 hours. The poly-aspartic and poly-aldehyde dextran is dialysed 10 times against distilled water at a ratio of 1/100.

Modification of the aminated plate with Aspartic-Aldehyde-Dextran

To 20 ml of aspartic-aldehyde-dextran, 1 ml of phosphate buffer 500 mM pH 7 is added followed by trimethylaminoborane in powder form until a concentration of 150 mM is reached. This solution is stirred with a magnetic stirrer for 45 minutes. Then, 350 μl are added to each well of an aminated plate (for example, those supplied by Costar), and they are left to react for 24 hours at room temperature. After this time, the plate is emptied and dried by holding it upside down and tapping it over filter paper. Finally, the bonds formed between the dextran and the plate are reduced (forming very stable secondary amino bonds), along with the remaining aldehyde groups (forming very inert hydroxyl groups) by adding 350 μl of carbonate buffer, 500 mM, pH 10.5, containing 10 mg/ml of sodium borohydride, to each well. After 30 min, the plates are washed with plenty of water, and dried by holding them upside down and tapping them over filter paper.

Immobilisation of the Aminated Oligonucleotide.

The aspartic-aldehyde-dextran immobilised on the plate is oxidised once more by adding 100 μl of sodium periodate 50 mM to each well. After 1 hour at room temperature, the plates are washed with plenty of distilled water. Next, 15 μl of phosphate buffer 65 mM, pH 7.0, containing 369 ng of probe aminated at the 3′ terminus and NaCl 330 mM, are added to each well. The immobilised dextran and the probe are allowed to react by incubating the plates, sealed with adhesive paper, in an oven at 55° C. for 72 hours with gentle shaking. After this time, the plates with the immobilised probe are dried by holding them upside down and tapping them over filter paper, and reduced by adding 350 μl of sodium borohydride (100 mg/ml) in carbonate buffer 500 mM, pH 10.5, and letting stand for 1 hour at room temperature. Finally, the plates are washed with distilled water and dried by holding them upside down and tapping them over filter paper.

Results of Hybridisation

The conditions and samples of hybridisation were the same as those described in the previous example. The results obtained were as follows:

| | Signal | |
|---|---|---|
| Dilution of amplificate | Commercial plate | New plate |
| 1/25 | 3.46 | 2.87 |
| 1/125 | 3.303 | 2.37 |
| 1/625 | 1.333 | 1.05 |
| 1/3125 | 0.8 | 0.18 |
| Non-complementary DNA (1/25) | 0.18 | 0.417 |

We can therefore conclude that the probe immobilised on the plate is able to hybridise with its complementary DNA, detecting quantities of DNA of 1.2 ng/μl. However, when incubation is performed with amplified labelled DNA that does not contain the complementary sequence, at the high concentration of 30 ng/μl, there is still a significant non-specific retention due to ionic interactions with the support. However, the signal obtained is much lower than in the previous example (0.41), indicating that the introduction of negative charges in the dextran reduces but do not completely remove the non-specific interaction of the DNA of the sample with the support. Examples 3 and 4 describe the complete elimination of these interactions.

### EXAMPLE 3

Immobilisation of Aminated Oligonucleotides on Aminated Plates Doubly Activated with Aspartic Dextran of 2,000,000 D and Aspartic Dextran of 298,000 D.

Preparation of Aspartic-Aldehyde-Dextran:

Two dextrans, one with a molecular weight of 2,000,000 D and the other with a molecular weight of 298,000 D, were activated separately following the protocol described in Example 2.

Modification of the Aminated Plate with Aspartic-Aldehyde-Dextran

Firstly, the aspartic-aldehyde-dextran of a molecular weight of 2,000,000 D was immobilised, following the protocol described in Example 2. Subsequently, and following the same protocol, the aspartic-aldehyde-dextran of a molecular weight of 298,000 D was immobilised.

Immobilisation of the Aminated Oligonucleotide

The protocol described in Example 1 is followed exactly.

Results of Hybridisation

The conditions and samples of hybridisation were the same as those described in the previous example. The results obtained were as follows:

| | Signal | |
|---|---|---|
| Dilution of amplificate | Commercial plate | New plate |
| 1/25 | 3.46 | OUT |
| 1/125 | 3.303 | 2.88 |
| 1/625 | 1.333 | 1.256 |
| 1/3125 | 0.8 | 0.855 |
| Non-complementary DNA (1/25) | 0.18 | 0.25 |

In this case, hardly any signal is obtained from hybridisation with a DNA not complementary to the immobilised probe, indicating that the non-specific retention obtained in the previous case has almost completely disappeared. However, the specific hybridisation remains the same as Example 3, with a sensitivity limit of 0.24 ng/μl (Dilution 1/3125). Furthermore, if we compare the sensitivity obtained with the conventional method of hybridisation in plates that have probes immobilised using streptavidine-biotin, the sensitivity is the same.

As a demonstration of the strength of the immobilising covalent bonds, these plates reproduced the hybridisation results after being boiled in saturated NaCl solution for 20 minutes. This indicates that a very strong covalent immobilisation has been obtained of the aminated probe and that this probe is functional for hybridisation with DNA in solution that contains the complementary sequence

### EXAMPLE 4

Immobilisation of Aminated Oligonucleotides on Aminated Plates Doubly Activated with Aspartic Dextran of 2,000,000 D and Blocked with Anhydride Acetic

Preparation of Aspartic-Aldehyde-Dextran:

Dextrans, with a molecular weight of 2,000,000 D was activated separately following the protocol described in Example 2.

Modification of the Aminated Plate with Aspartic-Aldehyde-Dextran

The aspartic-aldehyde-dextran of a molecular weight of 2,000,000 D was immobilised, following the protocol described in Example 2. After immobilisation, the remaining active groups were blocked by incubation with a solution of 10% Acetic Anhydride in acetonitrile for 10 min, and finally the plate was washed with water and dried.

Immobilisation of the Aminated Oligonucleotide

The protocol described in Example 1 is followed exactly,

Results of Hybridisation

The conditions and samples of hybridisation were the same as those described in the previous example. The results obtained were as follows:

Signal

| Dilution of amplificate | Commercial plate | New plate |
|---|---|---|
| 1/25 | 3.46 | OUT |
| 1/125 | 3.303 | 2.936 |
| 1/625 | 1.333 | 1.32 |
| 1/3125 | 0.8 | 0.855 |
| Non-complementary DNA (1/5) | 0.18 | 0.101 |

In this case, no signal is obtained from hybridisation with a DNA not complementary to the immobilised probe, indicating that the non-specific retention has completely disappeared. However, the specific hybridisation remains the same, with a sensitivity limit of 0.24 ng/μl (Dilution 1/3125). Furthermore, if we compare the sensitivity obtained with the conventional method of hybridisation in plates that have probes immobilised using streptavidine-biotin, the sensitivity is the same

As a demonstration of the strength of the immobilising covalent bonds, these plates reproduced the hybridisation results after being boiled in saturated NaCl solution for 20 minutes.

Thus, in the present invention, the use of dextrans or other polysaccharides as long, inert and hydrophilic spacer arms is demonstrated for improving the methods of covalent immobilisation of DNA probes (or other ligands of interest) at surfaces of interest in clinical diagnostics. Such surfaces include microtitre plate walls, magnetic particles, micro-arrays, biosensors, etc. An example of these new methods constitutes the use of hetero-poly-functional polymers containing for example aspartic acid and aldehyde groups for the activation of aminated commercial plates and their subsequent use for the appropriate immobilisation of DNA probes that contain an amino group bound at its 3′ or 5′ terminus. Thus, it is possible to achieve a covalent immobilisation of DNA probes at one of the termini of the sequence by means of very stable chemical bonds (secondary amines). It is also possible to ensure that the immobilised DNA probes are kept at a distance from the surface of the plate to prevent steric restrictions for the hybridisation of complementary soluble DNA. The use of dextrans with negative charges as spacer arms also makes it possible for the immobilised DNA probe to be almost completely inert against possible non-specific adsorption of soluble DNA. Even more interestly, the final blocking of the plates with acetic anhydride enable to fully prevent even traces of non-specific DNA The new plates of DNA immobilised in this fashion allow experiments of hybridisation to be performed in drastic experimental conditions (for example, in the presence of formamide or hybridisation temperature higher than 60° C.) in which conventional probes (bound to the microtitre plates by steptavidin-biotin complexes) are not stable. Performing these highly astringent hybridisation experiments allows us to avoid all hybridisations where there is not an exact correspondence of all bases of the probe and the hybridised DNA. In this fashion, it is possible to detect mutations of a single base in genes that are relevant as possible markers of disease.

1. An oligonucleotide immobilised to a surface by a hetero-poly-functional saccharide or related polymer.

2. An oligonucleotide according to claim 1, wherein the saccharide is a dextran.

3. An oligonucleotide according to claim 1 or 2, wherein the saccharide is bifunctional.

4. An oligonucleotide according to any preceding claim, wherein the saccharide has am anionic function.

5. An oligonucleotide according to any preceding claim, wherein the saccharide has a covalently reactive function.

6. An oligonucleotide according to any preceding claim, wherein the surface is chosen from microtitre plates, magnetic particles, agarose supports, epoxy-acrylic resins, nylon, glass surfaces, or gold surfaces.

7. A procedure for immobilisation of an oligonucleotide where the oligonucleotide is immobilised to a surface using a hetero-poly-functional polymer.

8. A method of detection which uses an oligonucleotide according to any of claims 1 to 6.

9. A method to detect mutation in a sample of oligonucleotide by hybridisation to an immobilised oligonucleotide which employs immobilised oligonucleotide according to any of claims 1 to 6.

10. A method according to claim 9, wherein the mutation involves a single base.

11. The use of a hetero-poly-functional polymer in the immobilisation of an oligonucleotide for use in a diagnostic method.
